(19) United States
(12) Patent Application Publication
(10) Pub. No.: US 2014/0343602 A1
(43) Pub. Date: Nov. 20, 2014

(54) EXPANDABLE OCCLUSION DEVICE AND METHODS

(71) Applicants: Brian J. Cox, Laguna Niguel, CA (US); Paul Lubock, Monarch Beach, CA (US); Richard Quick, Mission Viejo, CA (US); Robert Rosenbluth, Laguna Niguel, CA (US)

(72) Inventors: Brian J. Cox, Laguna Niguel, CA (US); Paul Lubock, Monarch Beach, CA (US); Richard Quick, Mission Viejo, CA (US); Robert Rosenbluth, Laguna Niguel, CA (US)

(21) Appl. No.: 14/239,129

(22) PCT Filed: Aug. 17, 2012
(86) PCT No.: PCT/US2012/051502

§ 371 Date: Jul. 29, 2014

Related U.S. Application Data
(60) Provisional application No. 61/636,392, filed on Apr. 20, 2012.
(60) Provisional application No. 61/525,680, filed on Aug. 19, 2011.

(51) Int. Cl. A61B 17/00

(52) U.S. Cl. CPC A61B 17/0057; A61B 2017/00575; A61B 2017/00623; A61B 2017/00606
USPC 606/215

(57) ABSTRACT

An occlusion device and method for occluding an undesirable passage through tissue, such as a septal defect, that provides an expandable cylinder or other structure that occludes the passage internally or by covering one or more openings to the passage. The occlusion device includes a wire lattice or mesh that expands from a contracted catheter-deliverable state to an expanded state that occludes the passage. The lattice or mesh has one or more layers, with layers that provide structural support to the device, and layers that provide a lattice braiding or pore sizes that promote further occlusion by a biological process, such as tissue ingrowth that further occludes the affected passage.

### CROSS-REFERENCE TO RELATED APPLICATION(S)

This application claims benefit of priority to U.S. Provisional Patent Application No. 61/525,680, filed Aug. 19, 2011, and U.S. Provisional Patent Application No. 61/636,392, filed Apr. 20, 2012, which are incorporated by reference in their entirety.

### TECHNICAL FIELD

The present technology relates generally to cardiovascular devices, implant delivery systems, and methods of using cardiovascular devices and delivery systems to treat structural and functional defects in the heart and circulatory system. More specifically, the present technology is directed to the occlusion of undesirable blood flow passages to repair or mitigate structural heart defects and/or diminished blood flow characteristics.

### BACKGROUND

The human heart and the circulatory system can have an undesirable blood flow passageway that requires treatment, such as a structural heart defect that interferes with the normal flow of blood. The passageway may be a natural defect or the result of disease or trauma. As an example, a healthy human heart is divided into four main blood containing chambers called the right and left atria and the right and left ventricles. The right heart, containing the right atrium and ventricle, are separated by a muscular wall or septum from the left heart, containing the left atria and ventricle. The right heart supplies blood to the lung (pulmonary) circulation for oxygenation, and the left heart supplies the subsequent oxygenated circulation to the body. In the fetal heart prior to birth, oxygenated blood is supplied by the mother and consequently, small openings are present in the fetal heart and major vessels to bypass the pulmonary circulation. Normally, these openings fuse or functionally close shortly after birth when the baby begins breathing. In congenital heart defects, however, these openings or in some cases other similar malformations fail to close properly, potentially causing a variety of cardiac and related problems, including: congestive heart failure, pulmonary hypertension, cryptogenic stroke, transient ischemic attack (TIA), clots, emboli, migraines, etc. Common structural heart defects include: atrial septal defects (ASDs) (as illustrated in FIG. 1), patent foramen ovale (PFO), ventricular septal defects (VSDs) (illustrated in FIG. 2), and patent ductus arteriosus (PDA). FIG. 1 shows: heart 900, right atrium 901, right ventricle 902, left atrium 903, left ventricle 904, atrial septal defect 905, blood flow from body 906, blood flow to lungs 907, and blood flow from lungs 908. FIG. 2 shows: heart 910, right atrium 911, right ventricle 912, left atrium 913, left ventricle 914, ventricular septal defect 915, blood flow from body 916, blood flow to lungs 917, and blood flow from lungs 918.

In ASDs, blood may flow from the left atrium through the interatrial septum to the right atrium causing the mixing of arterial and venous blood (shunting) and increased right atrial pressure, both which may be clinically significant. In PFOs (illustrated in FIG. 3), the tissue flap across the left atrial septum opening (the foramen ovale), does not fuse shut. As the pressure in the left atrium is typically higher than the right atrium, this flap usually remains functionally closed in the estimated 25 to 30% of the adult population with this condition. However, there is evidence to suggest that at times (e.g., during coughing) in certain of these patients the flap opens with subsequent shunting that may lead to some of the aforementioned adverse neurological and vascular events. FIG. 3 shows: heart 920, right atrium 921, right ventricle 922, left atrium 923, left ventricle 924, patent foramen ovale 925, superior vena cava 926, and inferior vena cava 927.

VSDs are collectively the most common type of congenital heart defects in which an opening is present in the interventricular septum between the right and left heart. This type of defect is not normal prior to birth but is estimated to be present in 0.2 to 0.4% of newborns. As in ASDs, this opening may close some time after birth but a persistent opening may allow undesirable shunting of arterial blood from the left ventricle to the right ventricle.

A short vessel called the ductus arteriosus, serves to shunt blood from the pulmonary artery to the aorta, as a means of protecting the right ventricle of the fetus from pumping against the high resistance of the uninflated lungs. This vessel normally closes shortly after birth. Failure of this vessel to close is called PDA, and may ultimately result in congestive heart failure.

In view of the significant health consequences related to the existence of an undesirable blood flow passage in the heart or the circulatory system, there is a need for devices and techniques for occluding such passages. It is believed that existing devices and techniques for occluding undesirable blood flow passages with implantable devices suffer from several drawbacks, including:

1) the inability to collapse and maintain device delivery flexibility sufficient to reliably navigate blood vessels through small diameter introducers,

2) inadequate provisions for accurate and controlled positioning and seating during percutaneous delivery,

3) inadequate design consideration for anatomical challenges such as variations in defect size, shape, tissue thickness, and proximity to vital structures such as the coronary sinus, etc.,

4) the insufficient sealing of the defect,

5) the inadequate fixation of the device,

6) inappropriate hemodynamic design and/or materials selection leading to excessive thrombus or thrombo-emboli,

7) structural fatigue failure of the components,

8) inadequate provisions for natural tissue ingrowth and healing following implant,

9) abrasion and/or erosion of tissue contacting the device due to improper sizing, porosity, and/or stiffness characteristics,

10) improper sizing that interferes with cardiovascular functions such as the impingement of the aorta in the treatment of PFOs, and

11) the formation of thrombi on surface protrusions such as hubs.

Accordingly, there is a need for devices and methods that address one or more of these deficiencies.

### BRIEF DESCRIPTION OF THE DRAWINGS

The accompanying drawings, which are incorporated herein and constitute part of this specification, illustrate embodiments of the present technology, and, together with the general description given above and the detailed description given below, serve to explain the features of the present technology.

FIG. 1 is a cross-section view of a human heart with an atrial septal defect.

FIG. 2 is a cross-section view of a human heart with a ventricular septal defect.

FIG. 3 is a cross-section view of a human heart with a patent foramen ovale.

FIG. 4 is a side view of an embodiment of an occlusion device in accordance with the present technology implanted at a cardiac defect.

FIGS. 5-9 are a side views of the occlusion device of FIG. 4 and an embodiment of a delivery device in accordance with the present technology.

FIG. 10 is a side view of an mandrel and braid for making a lattice in accordance with an embodiment of the present technology.

FIG. 11A is a schematic view of a lattice component of an occlusion device in accordance with an embodiment of the present technology.

FIG. 11B is an isometric image of the lattice component of FIG. 11A.

FIG. 11C is a schematic of an occlusion device including the lattice component of FIG. 11A.

FIG. 12 is a cross-section view of the embodiment of FIG. 11A.

FIGS. 13 and 14 are side views of alternative embodiments of occlusion devices in accordance with the present technology.

FIG. 15A is a cross-section view of additional embodiments of an occlusion device in accordance with the present technology.

FIG. 15B is a schematic of an embodiment of the layering of the embodiment of FIG. 15A.

FIG. 16 is a cross-section view of additional embodiments of an occlusion device in accordance with the present technology.

FIG. 17A-17D are partial cross-section views of alternative embodiments of occlusion devices in accordance with the technology.

FIG. 18 are cross-section views of an embodiment of an occlusion device and an embodiment of a delivery device in accordance with the technology.

FIG. 19 is an expanded view of a portion of the occlusion device and delivery device of FIG. 18.

FIG. 20 is a cross-section view of an alternative embodiment of an occlusion device in accordance with the technology.

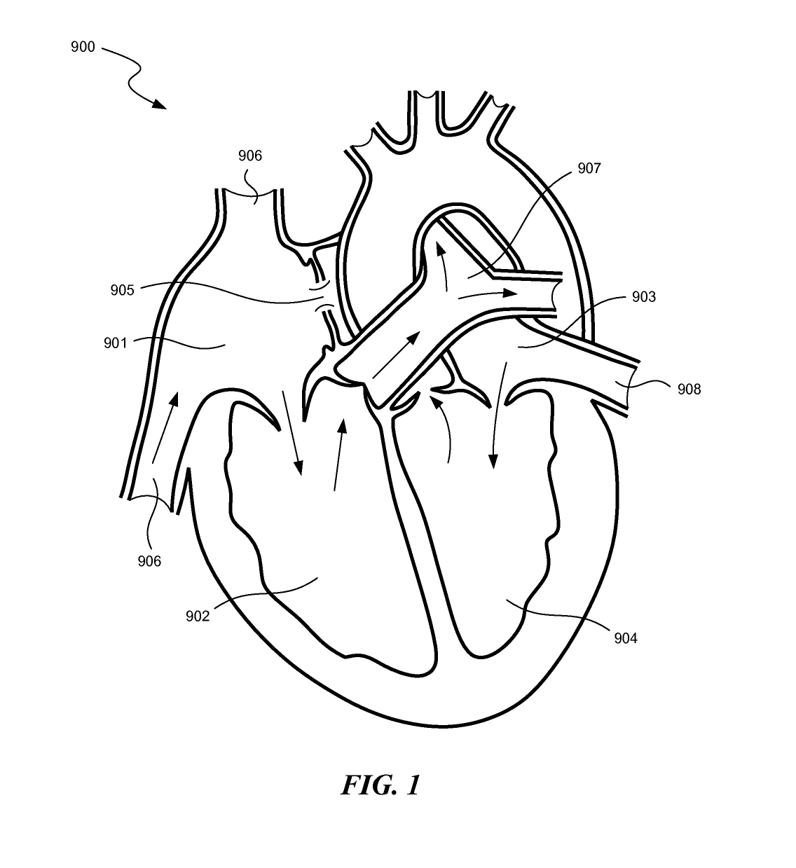

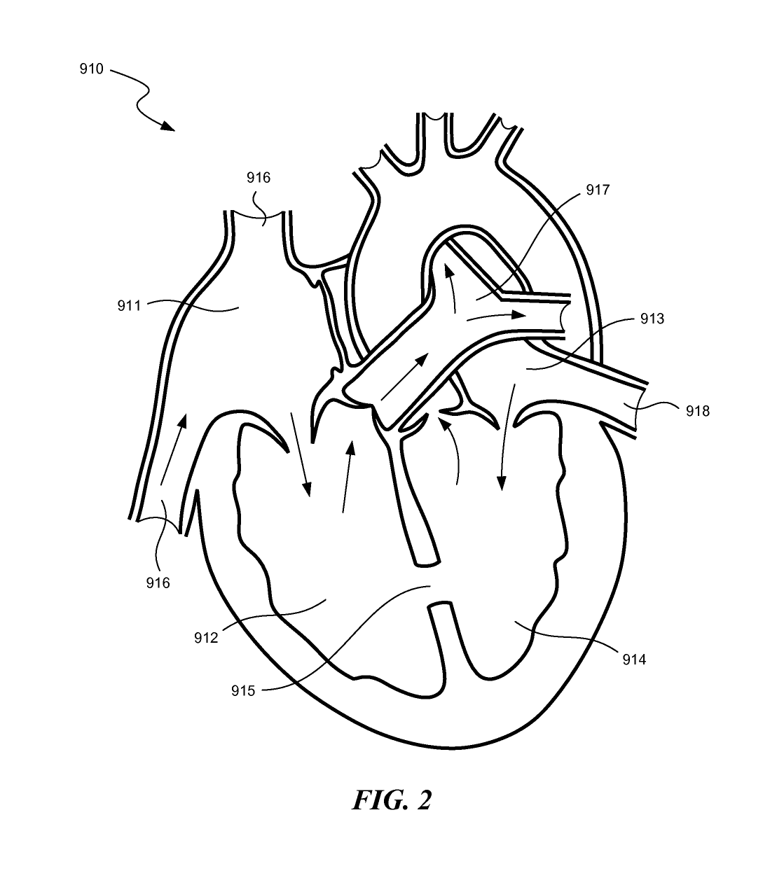

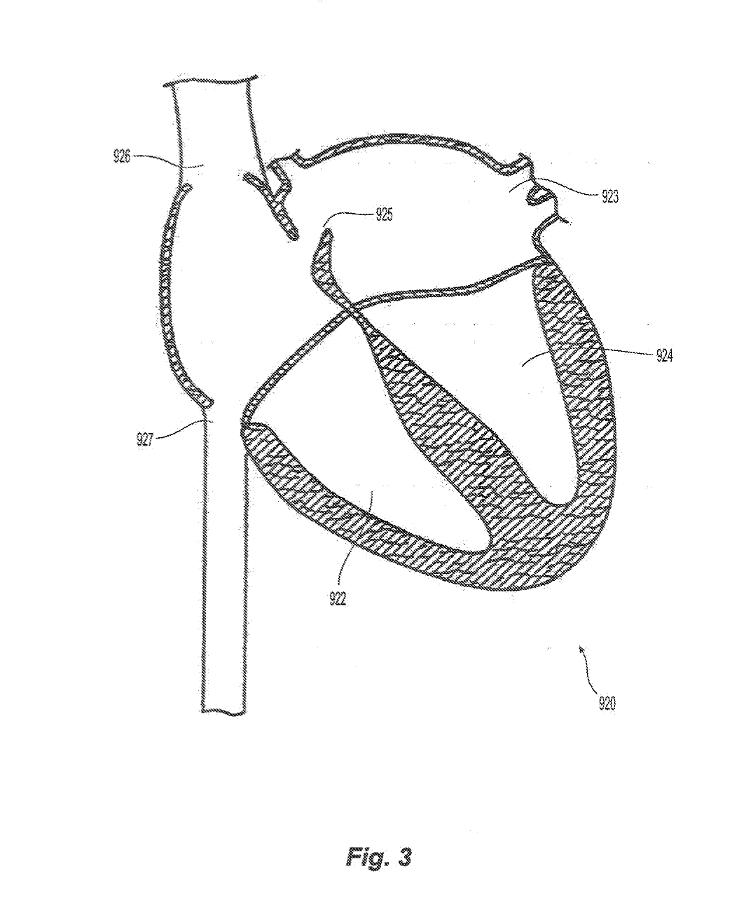

### DETAILED DESCRIPTION

Several embodiments of occlusion devices and delivery systems described herein implant a self-expanding occlusion member at a location where there is an undesirable passage within tissue, such as a blood flow passage extending into cardiac or vascular tissue. A “passage” as used herein includes an accessible opening within or through tissue, such as a two-ended passage connecting two portions of the cardiovascular system (e.g., a passage through a septum), a cul de sac or one-ended passage terminating within tissue (e.g., a left atrial appendage or an aneurysm), a passage exiting the cardiovascular system (e.g., a hemorrhage site), and/or an anatomical passage (e.g., a blood vessel, or a channel or duct of an organ). As described below, the occlusion member can occlude or at least partially occlude an undesired passage, and the structure and shape of the occlusion member can have multiple layers of at least one self-expanding lattice that controls the occlusion of the passage. The occlusion member itself can initially partially occlude the passage and then quickly induce a biological response that completely occludes the lattice.

### 1. Implementation of an Occlusion Device

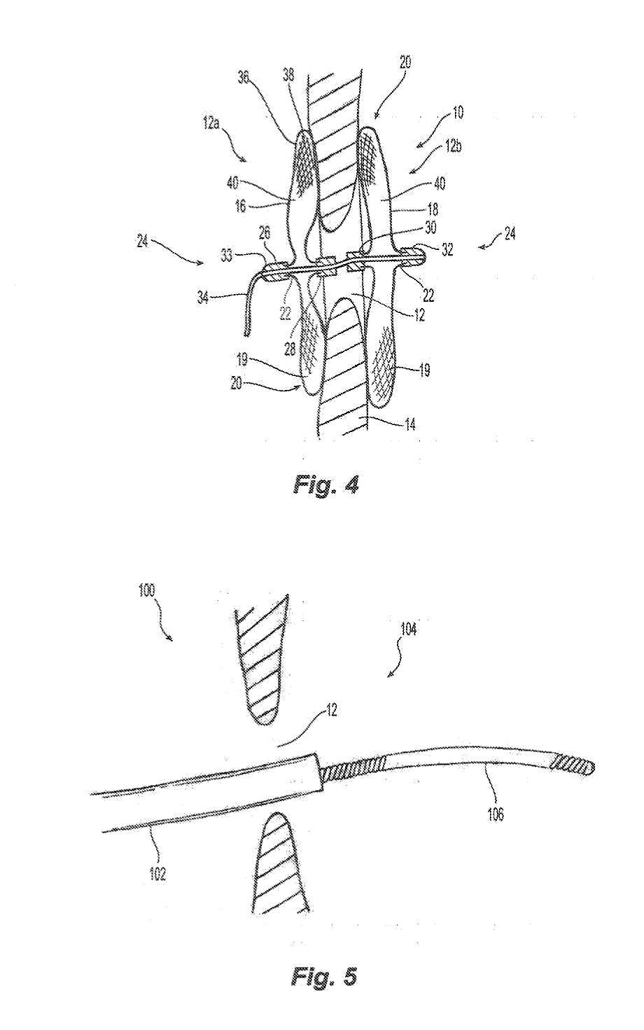

FIG. 4 is a side view of one embodiment of an occlusion device 10 having a first occlusion member 16 and a second occlusion member 18 implanted at a passage 12 through the septum 14 of the heart. The occlusion members 16, 18 can individually include a separate lattice structure 19. In several embodiments, the lattice structure 19 can have at least one wire mesh, such as a wire braid, that has a disc-shape after implantation. The first occlusion member 16 can further include outer and inner hubs 26 and 28, respectively, connected to the ends of the lattice structure 19 of the first occlusion member 16, and similarly the second occlusion member 18 can have outer and inner hubs 32 and 30 connected to the ends of the lattice structure 19 of the second occlusion member 18. Each of the hubs 26, 28 and 30 can have a channel 33. The occlusion device 10 can further include a tether 34 that passes through the channels 33 of the hubs 26, 28 and 30, and a distal end of the tether 34 can be attached to the outer hub 32 of the second occlusion member 18. In this particular embodiment, the lattice structure 19 of the first occlusion member 16 is separate from the lattice structure 19 of the second occlusion member 18.

After implantation, a peripheral portion 20 of the first occlusion member 16 contacts one side of the septum 14 to cover one open end 12a of the passage 12 and a peripheral portion of the second occlusion member 18 contacts the other side of the septum 14 to cover the opposite open end 12b of the passage 12. For example, the tether 34 can be pulled such that it slides through the hubs 26 and 28 to draw the first and second occlusion devices 16, 18 against the opposing sides of the septum 14. This causes the lattice structures 19 to press against the septum and cover the ends 12a and 12b of the passage 12.

In the embodiment of FIG. 4, with reference to only the first occlusion member 16, the lattice structure 19 can include at least one lattice layer 36 that extends across the entire surface of the first occlusion member 16 to occlude the passage 12. The wires or other types of cross members of the lattice structure 19 can be configured to provide pores 38 or other types of openings that promote further occlusion by a biological process on the lattice structure 19. The lattice layer 36, as shown, also defines an internal volume 40 of the first occlusion member 16 through which the tether 34 passes. As can be appreciated, the second occlusion member 18 illustrated in FIG. 4 can have the same structure as the first occlusion member 16.

### 2. Delivery Systems and Methods

Embodiments of delivery systems and methods for implanting the occlusion device 10 are illustrated in FIGS. 5-9. For clarity, FIGS. 5-9 show a partial cross-section view of the septum tissue 14 and side views of the first and second occlusion members 16, 18.

Referring to FIG. 5, one embodiment of a delivery system 100 includes a catheter 102 having a sheath 108 configured to be inserted over a pre-placed guidewire 106 to an implantation site 104. The guidewire 106 can have a lumen for receiving the tether 34 (FIG. 4), and the placement of the guidewire 106 and the subsequent insertion of the catheter 102 over the guidewire 106 can be performed using known imaging systems and techniques. In one example, the guidewire is inserted through the femoral vein or radial artery and advanced to the heart using an external imaging means, such as fluoroscopy, x-ray, MRI or the like, to direct the distal end of the guidewire to the passage to be occluded. As illustrated in FIG. 5, the guidewire 106 is positioned so that it extends through the passage 12. Radiopaque markers (not shown) can be incorporated into the guidewire, catheter, or the occlusion device itself to provide additional visibility under imaging guidance. Marker materials can include: tungsten, tantalum, platinum, palladium, gold, iridium, or other suitable materials. At this stage of the method, the first and second occlusion members 16 and 18 (not shown in FIG. 5) can be constrained in the sheath 108 in a low-profile or contracted state while the catheter 102 is advanced over the guidewire 106.

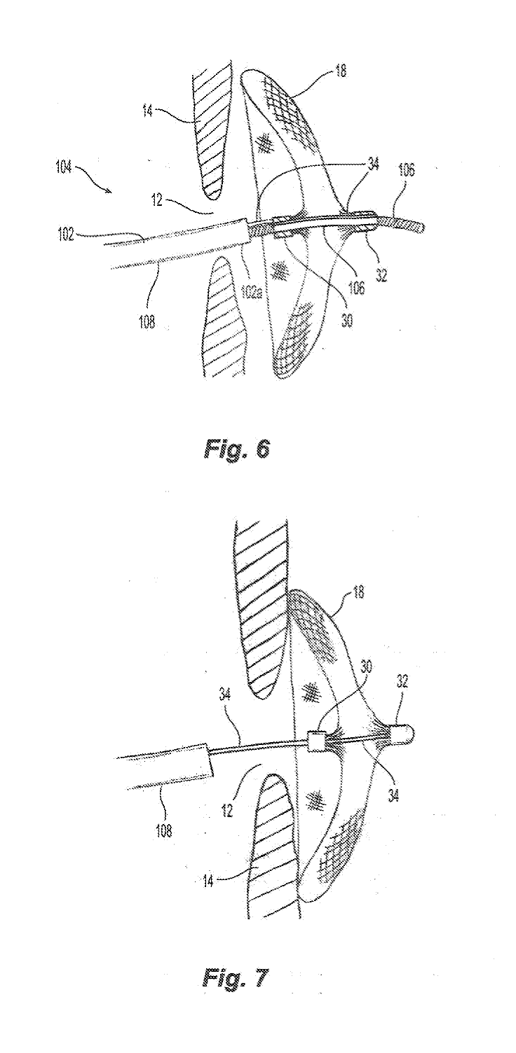

Referring to FIG. 6, after the distal end 102a of the catheter 102 is passed through the septum 14, the sheath 108 is withdrawn and/or the second occlusion member 18 is advanced to deploy the second occlusion member 18 on the distal side of the septum 14. For example, as sheath 108 is retracted in the proximal direction, the second occlusion member 18 in uncovered sufficiently to permit the lattice structure 19 to self expand.

Referring to FIG. 7, as the sheath 108 is pulled back, the tether 34 can remain engaged with the hub 32 to assist with keeping the second occlusion member 18 in place. Alternatively, the tether 34 can be introduced and secured to the second occlusion member 18 as the guidewire 106 is removed. After the second occlusion member 18 fully clears the sheath 108 and expands to a size greater than the passage 12, the guidewire 106 is then removed from the space to leave the tether 34 in place, as illustrated in FIG. 7. Tension can be applied to the tether 34 to seat the distal occlusion member 18 against the tissue 14 surrounding the passage 12. Alternatively, the tether itself can apply or contribute to the seating of the distal occlusion member 18 with a contracting force arising from the design or material properties of the tether, such as a tether made of a stretched elastic material, a tether with an extended tension spring structure, and/or a tether with shape memory properties configured to draw opposing hubs towards each other.

FIG. 8 shows a stage of the method after the sheath 108 has been further retracted in the proximal direction to uncover the first occlusion member 16 and hubs 26, 28. After expansion, the first occlusion member 16 is distally advanced to seat the first occlusion member 16 against the septum 14 surrounding the passage 12.

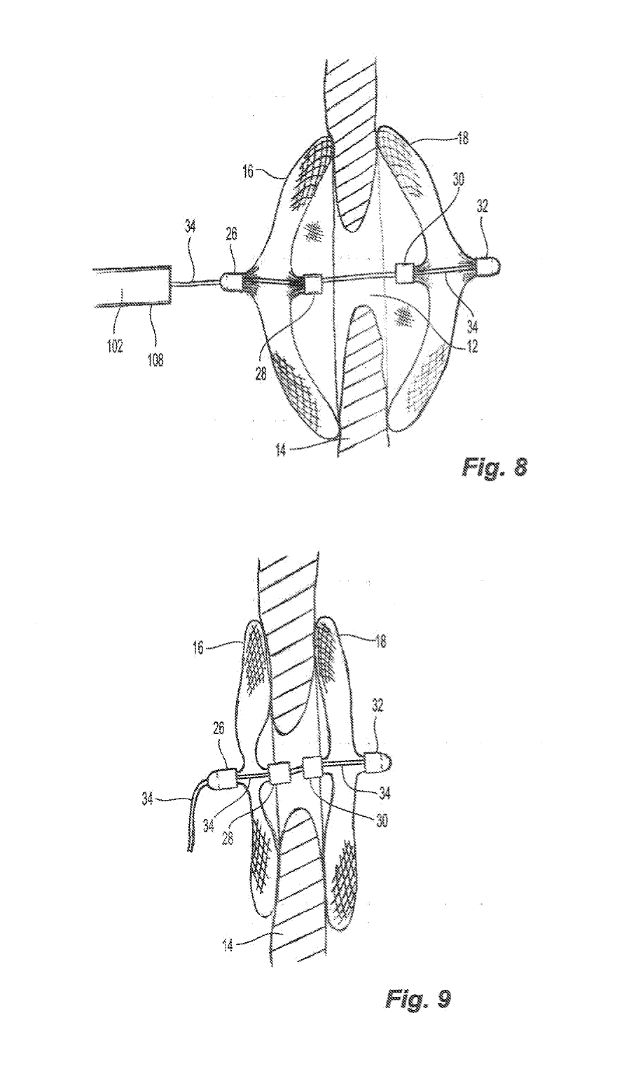

FIG. 9 shows a stage of the method after the first occlusion member 16 is subsequently moved distally closer to the second occlusion member 18 to effectively shortening the length of the portion of tether 34 extending between the inner hub 28 of the first occlusion member 16 and the inner hub 30 of the second occlusion member 18. The first occlusion member 16 can be advanced distally while tension is maintained on the tether 34 to cinch the first and second occlusion members 16, 18 together, thereby applying compressive force against the septum 14 surrounding the passage 12 and forming a substantial occlusion of the passage 12. The positions of the hubs 26, 28, 30, and 32 are subsequently fixed relative to the tether 34 to maintain the arrangement illustrated in FIG. 9. A retaining member (not shown) can be used to hold the first and second occlusion members 16, 18 in close apposition to the septum 14. For example, the retaining member can be component of the first occlusion member 16 or a separate component.

### 3. Lattice Structure and Formation

In any of the embodiments described herein, the occlusion members can have a lattice (e.g., a mesh) of wires, filaments, threads, sutures, fibers or the like, that have been configured to form a fabric or structure having openings (e.g., a porous fabric or structure). The lattice can be constructed using metals, polymers, composites, and/or biologic materials. Polymer materials can also include polymers such as Dacron, polyester, polypropylene, nylon, Teflon, PTFE, ePTFE, TFE, PET, TPE, PGA, PGLA, or PLA. Other suitable materials known in the art of elastic implants can be used. Metal materials can include, but are not limited to, nickel-titanium alloys (e.g. Nitinol), platinum, cobalt-chrome alloys, Elgiloy, stainless steel, tungsten or titanium. In some embodiments, it is desirable that the lattice be constructed solely from metallic materials without the inclusion of any polymer materials, i.e., polymer free. In these embodiments and others, it is desirable the entirety of the occlusion device be made of metallic materials free of any polymer materials. It is believed that the exclusion of polymer materials in some embodiments may decrease the likelihood of thrombus formation on device surfaces, and it is further believed that the exclusion of polymer and the sole use of metallic components can provide an occlusion device with a thinner profile that can be delivered with a smaller catheter as compared to devices having polymeric components.

The lattice can be a braided mesh of wires. The braided mesh can be formed over a mandrel as is known in the art of tubular braid manufacturing. The tubular braid can then be further shaped using a heat setting process. The braid can be a tubular braid of fine metal wires such as Nitinol, platinum, cobalt-chrome alloy, stainless steel, tungsten or titanium. The lattice can be formed at least in part from a cylindrical braid of elastic filaments. The braid can be radially constrained without plastic deformation and be self-expanding on release of the radial constraint. In several embodiments, the thickness of the braid filaments can be less than about 0.2 mm. For example, the braid can be fabricated from wires with diameters ranging from about 0.015 mm to about 0.15 mm.

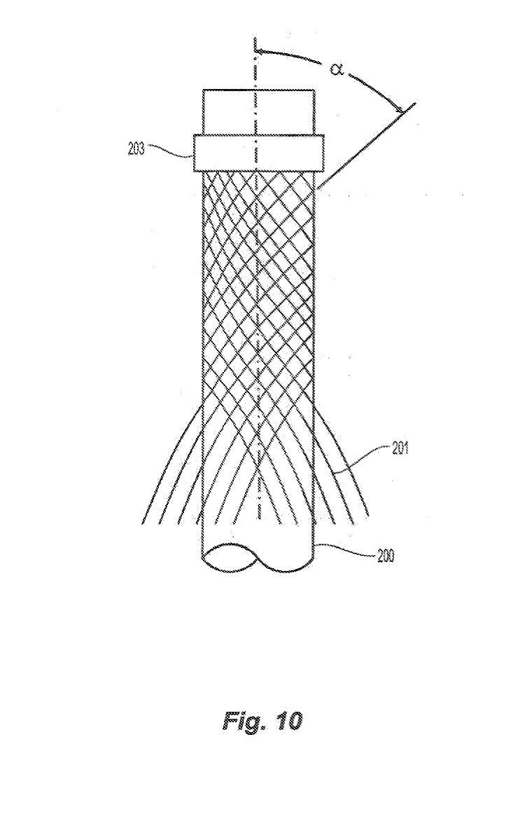

FIG. 10 shows a braided mesh being formed over a mandrel as is known in the art of tubular braid manufacturing. The braid angle alpha (α) can be controlled by various means known in the art of filament braiding. The braids for the mesh components can have a generally constant braid angle over the length of a component or can be varied to provide different zones of pore size and radial stiffness. The tubular braided mesh can then be further shaped using a heat setting process. Referring to FIG. 10, as is known in the art of heat setting a braiding filament 201, such as Nitinol wires, a fixture, mandrel or mold 200 can be used to hold the braided tubular structure 202 in its desired configuration while subjected to an appropriate heat treatment such that the resilient filaments of the braided tubular member assume or are otherwise shape-set to the outer contour of the mandrel or mold 200. The filamentary elements of a mesh device or component can be held by a fixture 203 configured to hold the device or component in a desired shape and, in the case of Nitinol wires, heated to about 475-525° C. for about 5-30 minutes to shape-set the structure. Other heating processes are possible and will depend on the properties of the material selected for braiding. For example, the braid can be a tubular braid of fine metal wires such as Nitinol, platinum, cobalt-chrome alloys, 35N L T, Elgiloy, stainless steel, tungsten or titanium. In some embodiments, the device can be formed at least in part from a cylindrical braid of elastic filaments. Thus, the braid can be radially constrained without plastic deformation and will self-expand on release of the radial constraint. The thickness or diameter of the braid filaments can be less that about 0.5 mm. The braid can be fabricated from wires with diameters or average diameters ranging from about 0.02 mm to about 0.40 mm. A device or component can have a high braid angle zone where the braid angle is greater than about 60 degrees. Such braids of shape memory and/or elastic filaments are herein referred to as “self-expanding.”

For braided portions, components, or elements, the braiding process can be carried out by automated machine fabrication or can also be performed by hand. For some embodiments, the braiding process can be carried out by the braiding apparatus and process described in U.S. patent application Ser. No. 13/275,264, filed Oct. 17, 2011 and entitled “Braiding Mechanism and Methods of Use” by Marchand et al., which is herein incorporated by reference in its entirety. In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance “d” from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

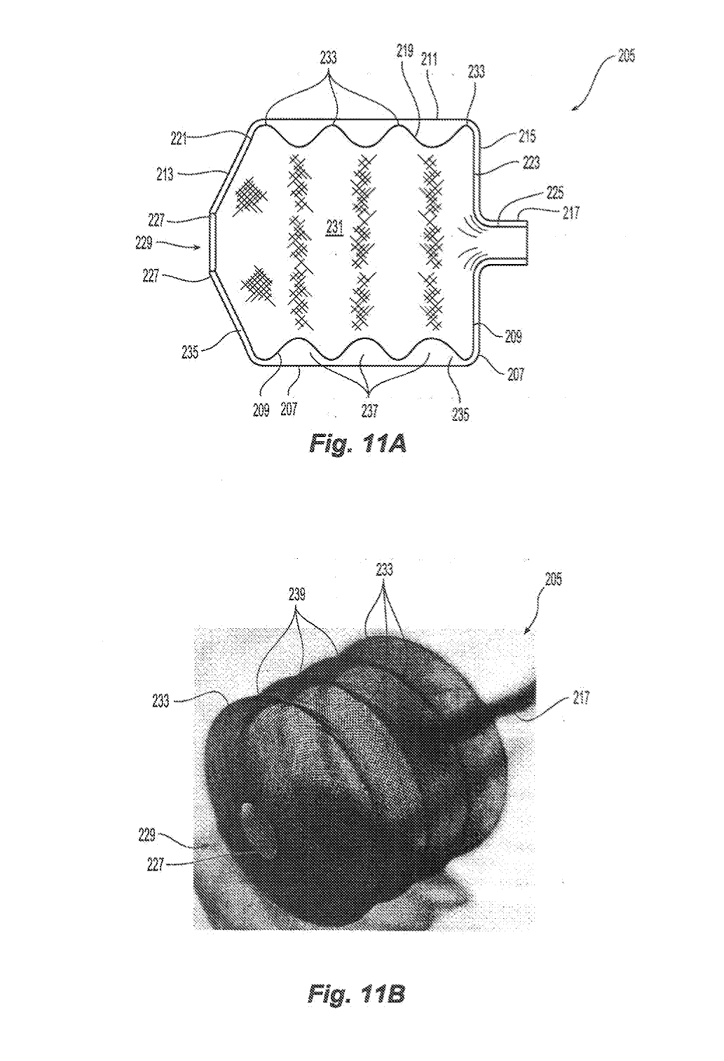

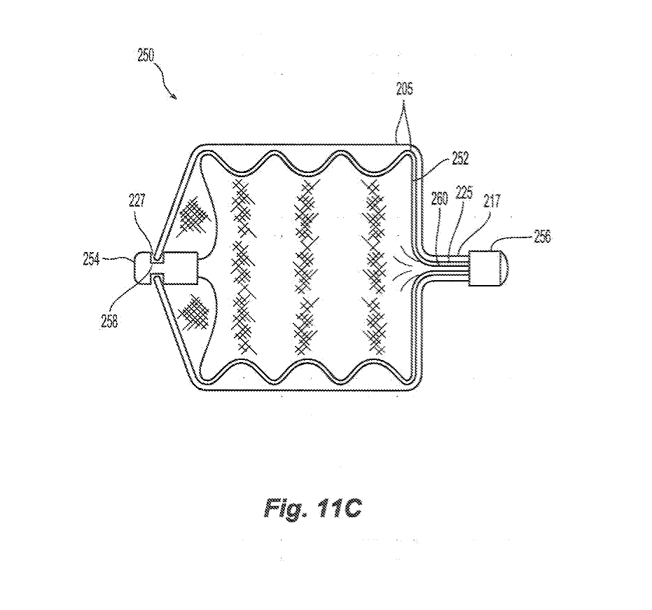

FIGS. 11A and 11B show an embodiment of a lattice or braid 205 that is a component of an occlusion device. As shown schematically in FIG. 11A, the lattice 205 has two layers with an external layer 207 and an internal layer 209. The external layer 207 has a cylindrically-shaped portion 211, a tapered portion 213, a planar portion 215, and gathered ends 217 of the lattice 205, and the internal layer 209 has an undulating portion 219, a tapered portion 221, a planar portion 223, and gathered ends 225 of the lattice 205. The external taper portion 213 and the internal taper portion 221 meet at an edge 227 where the lattice 205 is everted (folded back onto itself) to circumscribe an aperture 229 leading to an internal volume 231 of the lattice 205. In the illustrated embodiment, the lattice 205 has a tubular braid of “360 Nitinol” wires of 0.025 mm (0.001 inch) diameter. In the manufacture of the lattice 205, the tubular braid was heat set as described herein to form undulations 233 along the undulating portion 219 of the internal layer 209.

In the schematic of FIG. 11, a spacing 235 is shown between the external layer 207 and the internal layer 209 for clarity. It can be appreciated that the external layer 207 and the internal layer 209 may contact each other substantially and thus have no spacing at some locations, and that the contact between the external and internal layers 207 and 209 can allow for the deformation of these layers as they rest against each other, as shown in FIG. 11B where portions of the external layer 207 exhibit depressions 239 where portions of the external layer 207 extend between adjacent undulation 233. It can be further appreciated that the undulations 233 provide substantially closed ring volumes 237 that extend circumferentially between the external and internal layers 207 and 209.

Referring to FIG. 11A, the external layer 207 and the internal layer 209 are portions of the same braid or lattice 205. As described above, the braiding characteristics of the lattice 205 can remain constant as the braid continues around the everted portion at the edge 227, or be formed with two or more braiding techniques so that the braiding on the inside (for the inner layer 209) is different than the braiding on the outside (for the external layer 207), so as to achieve desired characteristics. For example, the braiding can change to provide differing braid angles or pore sizes.

FIG. 11C schematically shows the lattice 205 component installed in an occlusion device 250 as an external lattice 205 assembled with an internal lattice 252, a first hub 254 having an external groove 258, and a second hub 256. The external lattice 205 has ends 217 and 225 that are secured to hub 256, and the everted portion of lattice 205 at edge 227 is positioned within the external groove 258 of the first hub 254. The ends 217 and 225 can be secured to the hub 256 by a weld, and the edge 227 is secured to the hub 254 by the groove 258 and the sizing of the aperture 229 that keeps the edge 227 within the groove 258. A locking member (not shown) can be secured to the groove 258 of the hub 254 to secure engagement with the edge 227. The internal lattice 252 has first ends 260 secured to the first hub 254 and second ends 262 secured to second hub 256 by, for example, a weld. In the embodiment of FIG. 11C, the external lattice 205 is an external occlusion lattice that provides an occlusive layer, and the internal lattice 252 is a structural lattice configured to support the occlusion lattice. In an example, a lattice that has a braid of large wires (e.g., between about 0.050 mm and 0.50 mm) can be used as a structural lattice (e.g., internal lattice 252) in an occlusion device that is covered by an external lattice (e.g. external lattice 205) having an occlusion layer with a fine lattice or braid structure. By placing the structural support layer within the occlusion layer(s), the inner structural support layer can provide the majority of the radial force for facilitating a seal between the occlusion device and the tissue surrounding the passage to be occluded, e.g., a blood vessel, the left atrial appendage, or the septal wall.

Lattice layers or portions of lattice layers can be constructed to have small pores to function as highly occlusive elements of the occlusion device. A layer can have at least a portion of the layer with an average effective pore size between about 0.050 mm and about 0.300 mm. An occlusive layer can be used having a maximum effective pore size of between about 0.050 mm and about 0.250 mm. Layers or portions of layers can be constructed to have large pores and function primarily as structure support and to provide radial force to facilitate conformance of other layers to surrounding tissue structures and thereby provide a seal between the device and tissue. The radial force provided by a structural component or layer can also inhibit movement, dislodgement and potential embolization of the device. A structural component or layer can have a maximum effective pore of between about 0.20 and 1.50 mm. The occlusion device can have one or more structural lattice layer(s) with a large (e.g., greater than about 0.250 mm) maximum effective pore size and one or more occlusive lattice layer(s) with a substantially smaller maximum effective pore size. The ratio of the maximum effective pore size of a structural lattice layer to an occlusive lattice layer can be between about 1.5 and 6. The difference between the maximum effective pore size of a structural lattice layer and the maximum effective pore size of an occlusive lattice layer can be between about 0.100 and 0.800 mm. The maximum effective pore can be determined by measuring more than about 5 pores around the periphery of the occlusion device where the pores tend to reach a maximum and averaging the numbers.

The shape and porosity of the lattice work together synergistically to provide defect occlusion and a biocompatible scaffold to promote new tissue ingrowth, neo-endothelialization, or healing tissue that substantially spans the lattice pores (the scaffold openings) of the braid. The tissue may span directly across the lattice pores from wire to adjacent wire to form a substantially smooth surface. Tissue may form substantially tangential to the lattice wires. The occlusive wire lattice may provide a matrix for healing without substantial involvement of an underlying sublayer. These functions can be influenced by the “pore size” or “weave density” of the lattice. It is believed that the lattice provides higher wire counts than current heart defect devices and thus smaller pore sizes that yield improved occlusion performance and possibly obviate the need for polymer fabric components that can increase thromboembolic risk. Pore sizes in the range of about 0.10 mm to 2.0 mm can be utilized in the lattice. The pore size can be in the range of 0.20 mm to 0.75 mm.

The wires of the lattice can have diameters or average diameters when two or more sizes of wire are used in a single lattice layer. An occlusive lattice layer can have wires with an average diameter less than 0.4 mm. A structure lattice layer can have wires with an average diameter between about 0.07 mm and about 0.20 mm. In addition, a ratio can be defined by comparing the diameters or average diameters of the structural lattice layer wires to the diameters or average diameters of the occlusive lattice layer wires. Such a ratio of structural to occlusive lattice layer wire diameters or average wire diameters can be in an inclusive range from 2:1 to 12:1.

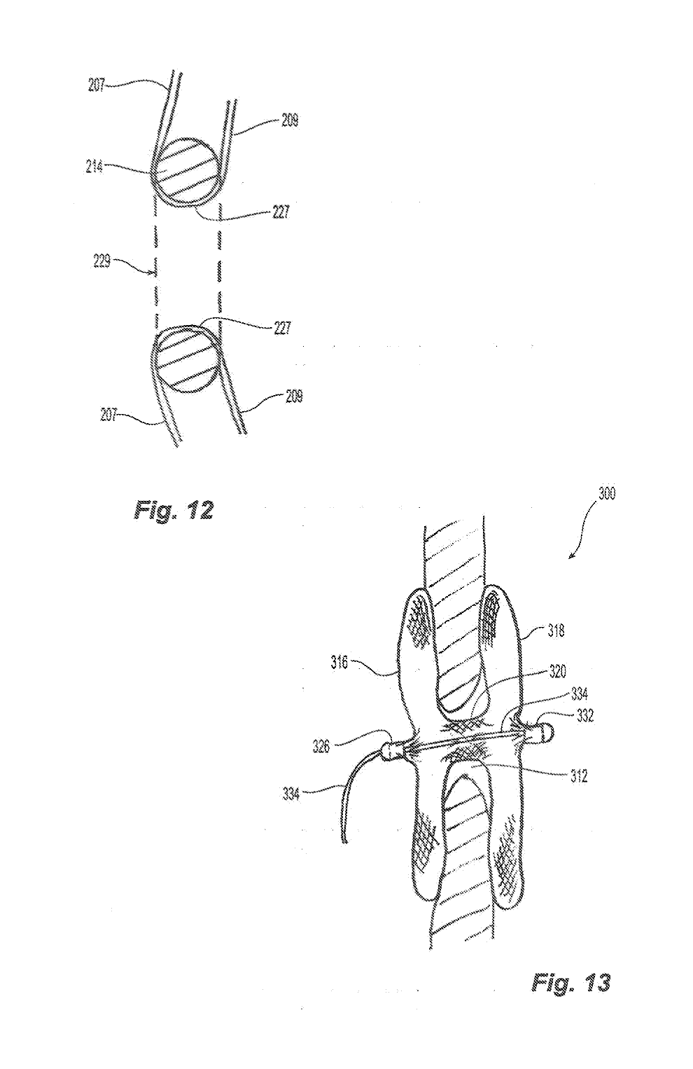

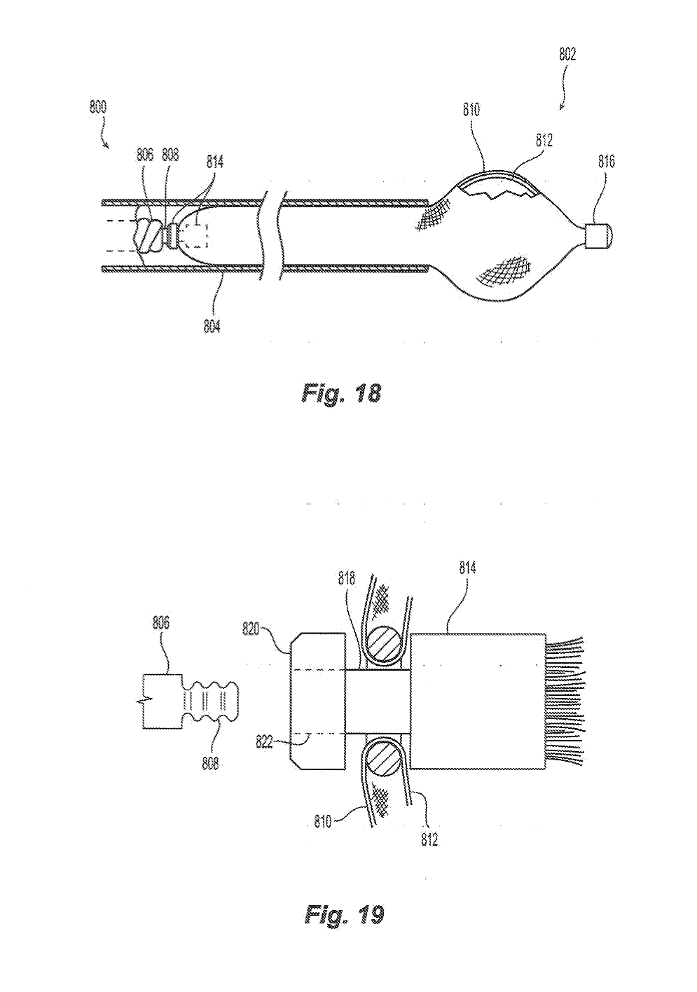

FIG. 12 shows a close view of a cross-section of an occlusion member where the lattice layer is everted. With reference to FIG. 11A, FIG. 12 shows external layer 207, internal layer 209, the edge 227, and the aperture 229. Also shown is an internal ring member or loop 214 that is provided between the internal and external layers 207 and 209 to support the shape of edge 227 and maintain the shape of the aperture 229. The loop 214 can be a series of loops such as, for example, a series of loops formed by wire thread wrapped around the edge 227 and tied off. FIG. 18 (described below) also shows the edge and loop in place about a hub. The loop 214 can be fixed to the external or internal layers 207 and 209 or trapped within the lattice when the lattice is everted.

### 4. Occlusion Member Shapes and Layering

The occlusion member can have various geometries depending on the application. For example, the occlusion member can include one or more layers of the same lattice material or different lattice materials having a generally cylindrical, spherical, ellipsoidal, oval, barrel-like, conical, frustum or other geometric shape. The layers of the lattice can have different shapes, such as an undulated or wave-like portion that serves as a flow baffle and/or conformal sealing layer, or a saw-toothed or bellows-like portion. The lattice layers can be heat set to form radial undulations, diameter changes, wrinkles, dilations or the like to form baffles or compartments. For example, the undulations can have sinusoidal-like undulations.

The lattice of the occlusion member can have a single layer of latticed or braided wires or provide a multilayer lattice. Two layers can be formed from one tubular braid that has been everted or folded back on itself to form a two-layer construct as describe above with regard to FIGS. 11A, 11B, and 12. An everted mesh forming two layers can be either in innermost layers, intermediate layers or outermost layers of the occlusion member. The layers can be configured in a substantially coaxial fashion. The layers or some of the layers can be held at one or more ends by a common connecting member or hub. One or more of the layers can have an open end that is not held by a connecting member or hub. An unfixed end of the layer can allow different lengths without bunching of the layers upon collapse for delivery or retraction by a catheter, as the layers can move relative to each other to accommodate the compression of the occlusion member into a contracted state. The occlusion member can have one undulated wave or bellows-like shape between two cylindrical lattice layers (see FIGS. 11A and 11B) such that the apices of the undulations touch or nearly touch the inner and outer cylindrical layers to form a plurality of substantially closed ring volumes.

Several configurations of occlusion member structure and shape, and lattice layering, are described in the following embodiments. As can be appreciated, the described features or combination of features for a particular embodiment can be applied to another embodiment. Furthermore, for clarity, features that are common to earlier-described embodiments are not again described in detail, as reference can be made to those earlier descriptions.

FIG. 13 illustrates an embodiment of an occlusion device 300 that is similar to the embodiment of the occlusion device 10 illustrated in FIG. 4. The occlusion device 300 does not have two facing hubs 28, 30 (FIG. 4) of the occlusion device 10, but instead the occlusion device 300 has a proximal occlusion member 316, a distal occlusion member 318, and an interposed lattice core 320 that provide a lattice layer within the passage 312. The proximal occlusion member 316, distal occlusion member 318, and lattice core 320 can be formed from a common lattice structure having one or more layers of the same or different lattices. The occlusion member 300 can further include a proximal hub 326, distal hub 332, and a tether 334 attached to the distal hub 332 such that the proximal and distal hubs 326 and 332 can be drawn together by the proximal retraction of tether 334.

During implantation the distal occlusion member 318 is deployed so that the lattice core 320 is disposed in the passage 312 and then the proximal occlusion member 316 is deployed. The expansion of the proximal and distal occlusion members 316 and 318 holds the lattice core 320 within the passage 312. The lattice provided in the occlusion members 316 and 318, and in lattice core 320 can be a continuous lattice layer or layers that extend from the proximal hub 326 to the distal hub 332. The occlusion member can also have a lattice in proximal occlusion member 316 that is distinct from the lattice of distal occlusion member 318, arranged so that portions of the lattices of both the proximal and distal occlusion members 316, 318 overlap or interweave to provide the lattice present in the lattice core 320.

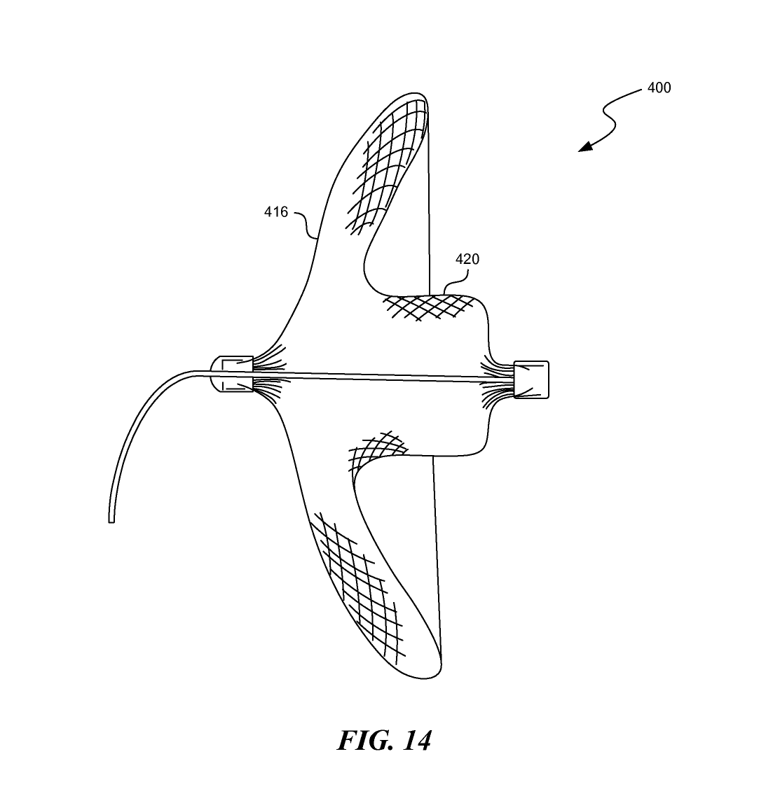

FIG. 14 illustrates another embodiment of an occlusion device 400 including an occlusion member 416 and a lattice core 420 that is configured for placement within a passage. In this embodiment, the occlusion member 416 can be used by itself or in conjunction with a second occlusion member (now shown in FIG. 14) similar to the second occlusion member 18 shown in FIG. 4.

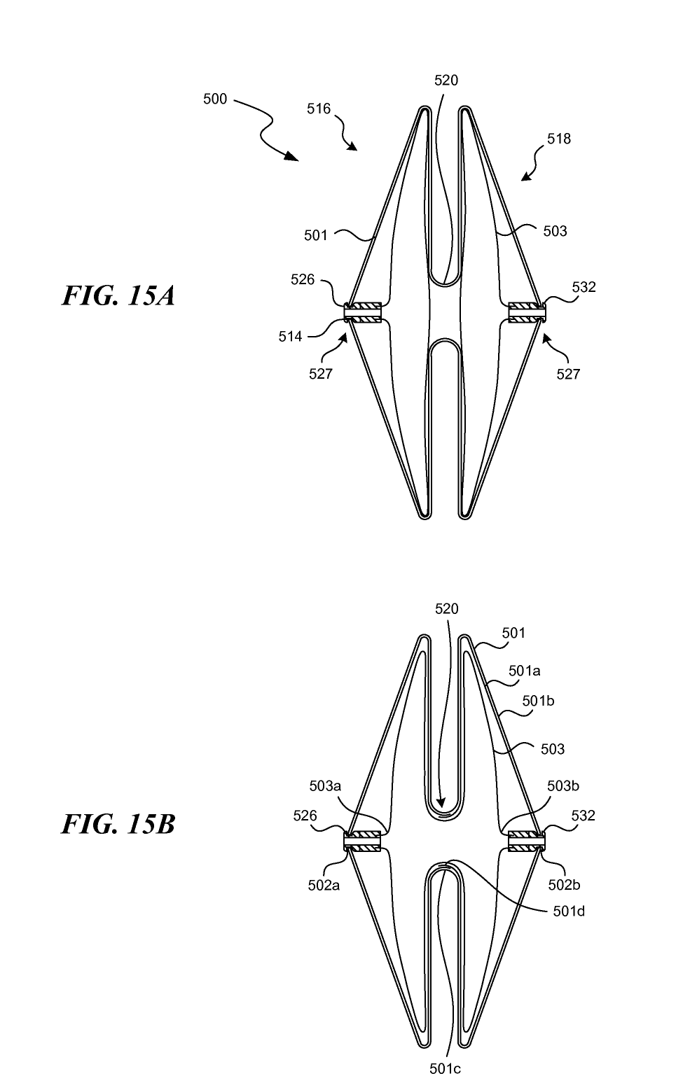

FIG. 15 illustrates another embodiment of an occlusion device 500 that includes a proximal occlusion member 516, a distal occlusion member 518, and a core 520 between the proximal and distal occlusion member 516 and 518. The proximal and distal occlusion members 516 and 518 can have conical shapes with the peak of the proximal occlusion member 516 at a proximal hub 526 and the peak of the distal occlusion member 518 at a distal hub 532. The proximal and distal occlusion members 516 and 518 can be a continuous layer of a single lattice material or layers of the same or different lattice materials. For example, the proximal and distal occlusion members 516 and 518 can have overlapping layers, interweaving layers, or fixed connections of one layer to another.

In the illustrated embodiment, each occlusion member 516 and 518 has an occlusion lattice 501, which can be folded to form a two-layer occlusion lattice of the same material, and a support lattice 503 within the occlusion lattice 501. The occlusion lattice 501 can be a wire mesh (e.g., wire braid) having wires arranged to provide pore sizes sufficient to promote quick formation and ingrowth of cells on the occlusion members 516, 518. The support lattice 503 can be a wire mesh (e.g., wire braid) having wires arranged to provide structural support for the occlusion lattice 501.

In the illustrated embodiment, the support lattice 503 is attached to the inner portions of the proximal and distal hubs 526, 532. For example, the ends of wires of the inner lattice layer 503 can converge and be connected to the hubs 516 and 532. The outer lattice layer 501 can be fixed to the proximal and distal hubs 526 and 532 with a ring member 514 that secures an everted outer lattice layer 501 within an exterior groove 527 on the outer surface of hubs 526, 532. The hubs 526, 532 can have a profile that follows the profile of the outer lattice layer 501, so that the protrusion of the hub past the outer lattice layer 501 is minimized. FIG. 15B shows an embodiment of the layering of the occlusion device 500 illustrated in FIG. 15A, with the inner lattice layer 503 having ends 503a and 503b that engage the proximal and distal hubs 526 and 532, As shown, the outer lattice layer 501 has two everted layers, with first outer lattice layer 501a covered by a second outer lattice layer 501b. As shown, an end 501c of the outer lattice layer 501 begins at the core 520, passes through a first everted portion 502a where the first outer lattice layer 501a folds back on itself to provide the second outer lattice layer 501b, passes through a second everted portion 502b where the layer returns to a first outer layer 501a, and then returns to the core 520 where an end 501d of the outer lattice layer 501 lies in an overlapped or interweave engagement with end 501c at the core 520.

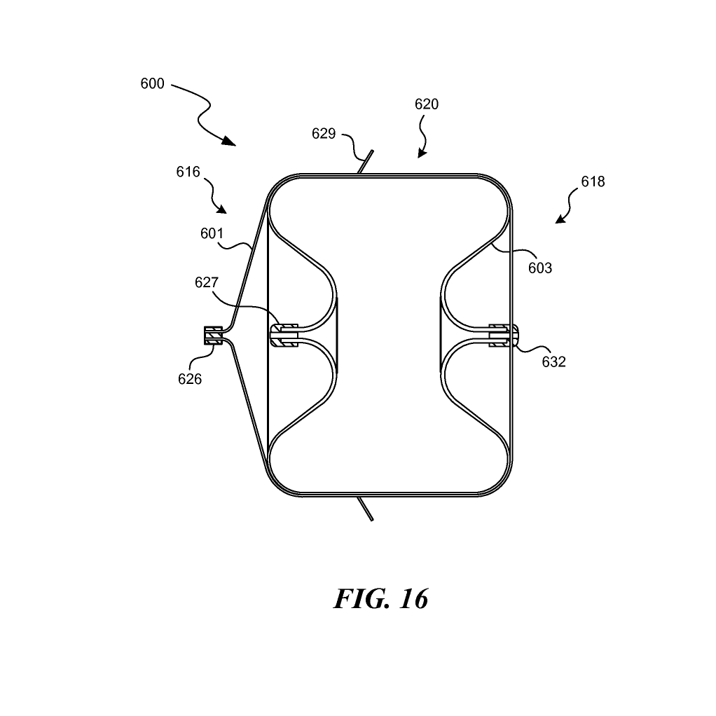

In an embodiment illustrated in FIG. 16, an occlusion device 600 has a proximal occlusion member 616 that has a conical shape and a distal occlusion member 618 that has a planar shape, and that are joined to each other by a lattice core 620 having an outer diameter that is equal to the outer diameters of the proximal and distal occlusion members 616, 618. Also illustrated is an outer lattice layer 601 (a two-layered everted layer) and an inner lattice layer 603 arrange so that the outer lattice layer 601 envelops inner lattice layer 603. Furthermore, the inner lattice layer 603 has wires and pore sizes sufficient to function as a structural lattice or braid disposed to support the shape and position of the outer lattice layer 601, and the outer lattice layer 601 has wires and pore sizes sufficient to function as an occlusive lattice or braid. Also illustrated are two proximal hubs, first proximal hub 626 and second proximal hub 627, and a distal hub 632, with the ends of wires of the inner lattice layer 603 converging to connect to the second proximal hub 627 and the distal hub 632. As can be appreciated, the inner lattice layer 603 includes undulations, and the non-connection between the two proximal hubs 626, 627 provides sufficient freedom of motion to accommodate compression of the occluding device 600 when it changes between the contracted state and the expanded state. If the inner and outer lattice layers were fixed at both ends, the two lattice layers can potentially interfere with each other when the device is expanded, unless the design of the layers anticipates that interference.

Also shown in FIG. 16 are retention members 629 that function as barbs to secure the occlusion device to the passage when implanted. The retention members 629 can be tines, barbs, hooks, pins or anchors that can be incorporated into the outer lattice layer 601 to help provide additional fixation of the occlusion device 600 to the heart wall or other tissue at or near the passage. The length of the retention members 629 can be from about 1 mm to 8 mm and preferably about 2 mm to 5 mm.

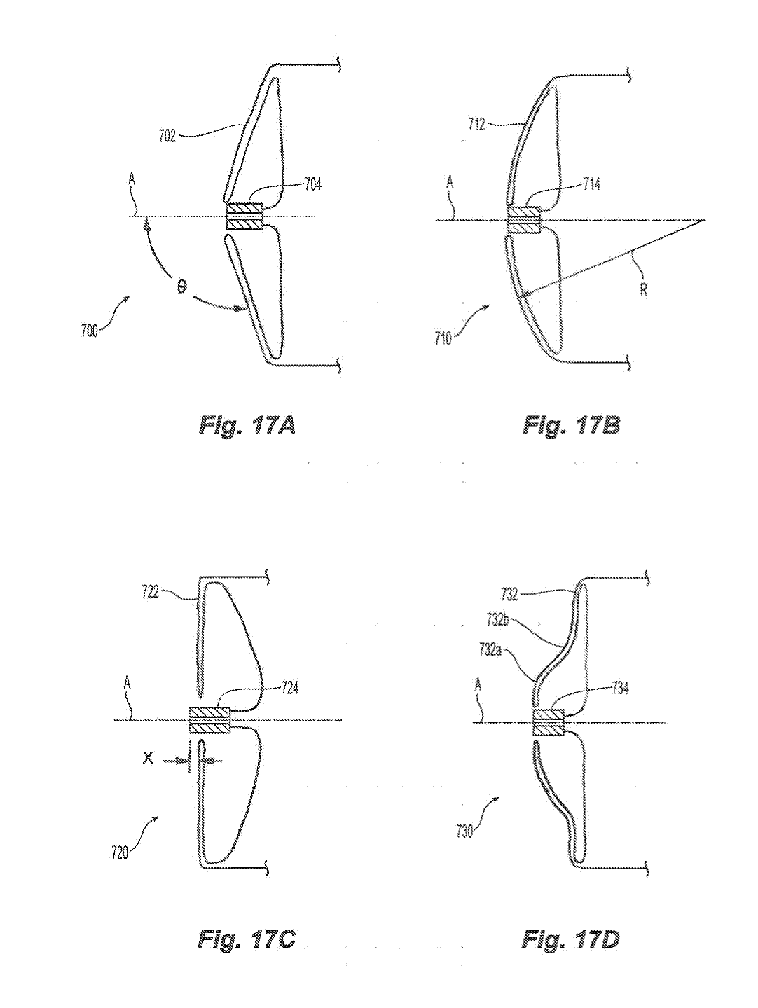

Alternative shapes for the ends of the occlusion device, at either the proximal or distal occlusion members are shown in FIGS. 17A-17D. As can be appreciated, these shapes can be applied to a single end of an occlusion device or to both ends, and the illustrated layers can be utilized in a multi-layer device. In any of the embodiments, the hub can be substantially enclosed or covered by the lattice layers so that a portion of the hub is not exposed from a viewpoint external to the expanded occlusion device and/or within a profile defined by the contour of the outer lattice layer. The hub and outer lattice layer can be configured so that at least 50% of the hub is enclosed or covered by the outer lattice layer or within a profile of the outer lattice layer. FIG. 17A illustrates an occlusion device 700 with an outer lattice layer 702 providing a conical shape where the inverted lattice outer layer 702 is disposed about a hub 704 that is recessed so that the hub does not protrude past the shape defined by the outer lattice layer 702, and illustrates a conical angle 8 of the conical shape that is between about 92 degrees and about 130 degrees and, more preferably, between about 100 degrees and about 120 degrees relative to an occlusion device axis A. FIG. 17B illustrates an occlusion device 710 with an outer lattice layer 712 providing an arcuate shape that with an inwardly facing curve have a radius, R, of between about 0.5 and 5 times the occluding device diameter. FIG. 17B also shows a hub 714 that is recessed relative to the arcuate shape of outer lattice layer 712. FIG. 17C illustrates an occlusion device 720 with an outer lattice layer 722 providing a planar shape that is substantially flat or disc shaped and disposed perpendicularly to the axis A of the occluding device 720, and illustrates a reduced-profile hub 724 that extends (as shown, extending from the lattice) or recedes (further into the lattice) from the profile of the lattice (here, a planar shape) by a distance X, which in an embodiment can be about 5 mm, and in another embodiment can be less than about 2 mm. FIG. 17D illustrates an occlusion device 730 with an outer lattice layer 732 providing an undulating shape having a combination of a convex surface 732a and a concave 732b surface, and illustrates a hub 734 that is flush with the profile of the arcuate shape.

FIGS. 18 and 19 show an embodiment of the delivery system 800 and a partially contracted occlusion device 802. As illustrated, the delivery system 800 is a catheter having a sheath 804, a rod 806, and a detachment system 808, and the occlusion device 802 has an outer lattice 810, an inner lattice 812, a first hub 814, and a second hub 816. As shown in FIG. 19, which is a closer view of a portion of FIG. 18, the hub 812 has a groove 818 holding the position of the inner and outer lattice 810 and 812, and has a locking member 820 that has a hole 822 with internal threads. The rod 806 at its end has a detachment system 808 providing screw threads that match the internal threads of the hole 822. As shown in FIG. 18, during deployment the detachment system 808 engages the locking member 820 to facilitate the deployment of the occlusion device 802. After deployment is completed, the detachment system 808 can disengage from the locking member 820 by unscrewing.

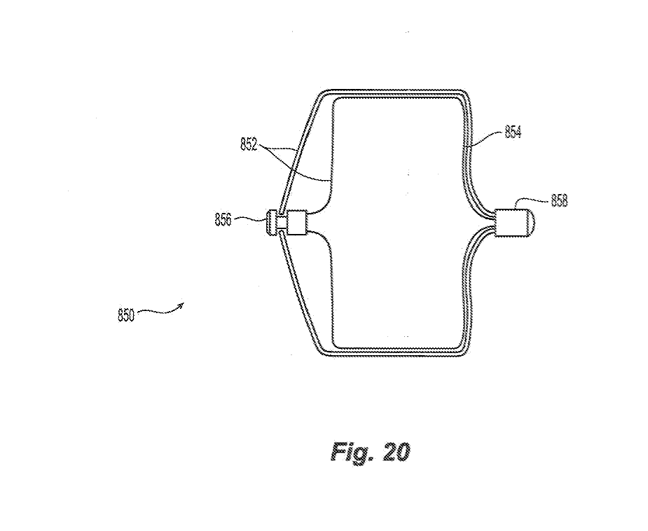

In some embodiments, such as shown in FIG. 19, the inner lattice layer is configured to collapse completely or nearly completely with the outer layer of the occlusion device. Complete collapse, that is collapse without bunching of either the inner or outer lattice layers, allows the device to be delivered through the smallest possible catheter as shown in FIG. 19. One way to collapse the occlusion device is to configure the outer layer to have substantially the same length as the inner layer(s). Alternatively, the inner and outer lattice layers can have different length by providing a braiding the mesh of one of the lattice layers (the inner or outer lattice layers) with a significantly larger braid angle than the mesh of the other lattice layer so that, when collapsed, one of the lattice layers substantially adjusts for any difference in lengths of the lattice layers. As an example, FIG. 20 shows a occlusion device 850 having an outer lattice 852, and inner lattice 854, a first hub 856, and a second hub 858. The outer lattice 852 has a substantially shorter total length, along the surface of outer lattice 852 from hub 856 to 858, in the expanded state than the inner lattice 854. As can be appreciated, when the occlusion device 850 is contracted into its delivery profile, the hubs 856 and 858 will move away from each other and the outer lattice 852, having a shorter total length, would appear to not be capable of allowing the hubs 856 and 858 to move apart sufficiently to allow the inner lattice layer 854 to flatten out. In the embodiment of FIG. 20, the outer lattice 852 is fabricated with a substantially larger braid angle than the inner lattice 854 that allows that outer lattice 852 to elongate to a length sufficient to accommodate the collapse of inner lattice 854 so as to avoid bunching of either lattice 852 or 854. In some embodiments, the braid angle of the inner lattice is between about 10° and about 30°. In some embodiments, the braid angle of the outer lattice is between about 25° and about 45°. In some embodiments, the braid angle of the outer lattice is between about 30% and 70% larger than the braid angle of the inner lattice.

In any of the described embodiments, the occlusion device can be constructed to provide the delivery of an elution or of one or more beneficial drug(s) and/or other bioactive substances into the blood or the surrounding tissue. The device can also be coated with various polymers to enhance performance, fixation and/or biocompatibility. The device can incorporate cells and/or other biologic material to promote sealing, reduction of leak or healing. In any of the described embodiments, the device can include a drug or bioactive agent to enhance the performance and/or healing of tissue contacting the device, including: an antiplatelet agent, including but not limited to aspirin, glycoprotein lib/lila receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. The device can also include coating or other application of an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

From the foregoing, it will be appreciated that specific embodiments of the present technology have been described herein for purposes of illustration, but that various modifications may be made without deviating from the spirit and scope of the invention. Accordingly, the invention is not limited except as by the appended claims.

1. A device for occluding a passage through cardiac or vascular tissue, the passage having opposing open ends, the device comprising:
expandable proximal and distal occlusion members aligned with each other to define a common axis about which the occlusion members can expand, the proximal and distal occlusion members each configured to assume a low-profile contracted state that fits in an intravascular catheter and an expanded state configured to cover one of the opposing open ends of the passage, the proximal and distal occlusion members each having a center portion disposed about the axis and a peripheral portion extending from the center portion, the peripheral portion of each occlusion member configured to contact the tissue surrounding one of the opposing open ends of the passage in the expanded state, the center portion of each occlusion member having a proximal end and a distal end disposed such that the distal end of the proximal occlusion member faces the proximal end of the distal occlusion member;
a proximal hub disposed at each of the proximal ends of the proximal and distal occlusion members;
a distal hub disposed at each of the distal ends of the proximal and distal occlusion members; and
a tether extending between the distal end of the proximal occlusion member and the proximal end of the distal occlusion member, the tether having an adjustable length configured to draw the proximal and distal occlusion members towards each other in the expanded state,
wherein the proximal and distal occlusion members each have an outer lattice layer enclosing an inner lattice layer, the inner and outer lattice layers each having a plurality of wires arranged to define pores of the respective lattice layer, the pores of the outer lattice layer having a pore size sufficiently small in the expanded state to promote further occlusion by a biological process.

2. A device for occluding a passage through tissue, the passage having at least one open end, the device comprising:
a plurality of wires forming an expandable lattice defining an axis of the device about which the lattice can expand, the expandable lattice configured to assume a low-profile contracted state and an expanded state, the expandable lattice further configured to have in the expanded state a peripheral portion disposed about the axis and configured to contact the tissue to occlude the passage,
wherein the plurality of wires are arranged to define pores having a pore size sufficiently small in the expanded state to promote further occlusion by a biological process on the expandable lattice.

3. The device of claim 2, wherein the expandable lattice comprises an outer lattice layer overlapping an inner lattice layer.

4. The device of claim 3, wherein the outer lattice layer defines pores of a first pore size and the inner lattice layer defines pores of a second pore size, the first pore size being sufficiently small in the expanded state to promote further occlusion by a biological process on the expandable lattice, the second pore size being greater than the first pore size.

5. The device of claim 2, the expandable lattice being a first expandable lattice, the device further comprising a second expandable lattice disposed on the axis, the second expandable lattice configured to have in the expanded state a peripheral portion disposed about the axis and configured to contact the tissue to occlude the passage, the first expandable lattice configured to contact in the expanded state the tissue at the at least one open end of the passage and the second expandable lattice configured to contact in the expanded state the tissue at another open end of the passage.

6. The device of claim 2, further comprising a hub engaging at least one of the plurality of wires of the expandable lattice.

7. The device of claim 6, further comprising a tether extending from the hub along the axis.

8. The device of claim 6, wherein the expandable lattice defines a profile of the device intersecting the axis, the hub disposed on the axis at a distance of about 5 mm from the intersection of the profile and the axis.

9. The device of claim 6, wherein the expandable lattice defines a profile of the device intersecting the axis, the hub disposed on the axis at a distance of less than about 2 mm from the intersection of the profile and the axis.

10. The device of claim 2, wherein the expandable lattice is configured to define a frusto-conical shape in the expanded state, the frusto-conical shape having a narrow end and an opposing wide end, the frusto-conical shape configured to dispose the wide end towards the tissue.

11. The device of claim 2, wherein the expandable lattice is configured to define an arcuate shape in the expanded state, the arcuate shape providing a curvature configured to curve towards the tissue, the curvature defining a radius centered on the axis.

12. The device of claim 2, wherein the expandable lattice is configured to define a planar shape in the expanded state, the planar shape having a central portion disposed on the axis, the planar shape extending perpendicularly from the central portion in a radial direction to the axis.

13. The device of claim 2, wherein the expandable lattice is configured to define an undulating shape in the expanded state.

14. The device of claim 3, wherein the outer lattice layer is an occlusive lattice layer having wires with an average diameter less than 0.4 mm.

15. The device of claim 14, wherein the inner lattice layer is a structural lattice layer having wires with an average diameter between about 0.07 mm and about 0.20 mm.

16. The device of claim 15, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

17. A device for occluding a passage through cardiac or vascular tissue, the passage having opposing open ends, the device comprising:
a plurality of wires forming an expandable lattice configured to change from a low-profile contracted state to an expanded state, the expandable lattice defining an axis of the device about which the lattice can expand, the expandable lattice in the expanded state having opposing covering members disposed on the axis and engaging each other via an occluding member, the occluding member being configured to occlude the passage in the expanded state and the covering members being configured to be radially larger than the occluding member to cover the opposing open ends of the passage in the expanded state,
wherein the expandable lattice has an outer lattice layer enclosing an inner lattice layer.

18. The device of claim 17, wherein in the expanded state the plurality of wires of the outer lattice layer define pores of the outer lattice layer and the plurality of wires of the inner lattice layer define pores of the inner lattice layer, a pore size of the inner lattice layer differing from a pore size of the outer lattice layer in the expanded state.

19. The device of claim 17, the device further comprising a hub disposed on the axis, the inner and outer lattice layers engaging each other via the hub.

20. The device of claim 19, wherein the hub fixedly engages the inner lattice layer.

21. The device of claim 19, wherein the expandable lattice defines a profile of the device intersecting the axis, the hub disposed on the axis at a distance of about 5 mm from the intersection of the profile and the axis.

22. The device of claim 19, wherein the expandable lattice defines a profile of the device intersecting the axis, the hub disposed on the axis at a distance of less than about 2 mm from the intersection of the profile and the axis.

23. The device of claim 17, wherein in the expanded state at least one of the covering members has a shape that is at least one of conical, frusto-conical, arcuate, planar, and undulating in a direction of the axis.

24. The device of claim 17, wherein the outer lattice layer is an occlusive lattice layer having wires with an average diameter less than 0.4 mm.

25. The device of claim 24, wherein the inner lattice layer is a structural lattice layer having wires with an average diameter between about 0.07 mm and about 0.20 mm.

26. The device of claim 25, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

27. A device for occluding a passage through tissue, the passage having at least one open end, the device comprising:
a plurality of metallic wires forming an expandable lattice defining an axis of the device about which the lattice can expand, the expandable lattice configured to assume a low-profile contracted state and an expanded state, the expandable lattice further configured to have in the expanded state a peripheral portion disposed about the axis and configured to contact the tissue to occlude the passage; and
a metallic hub engaging at least one of the plurality of wires of the expandable lattice,
wherein the plurality of wires are arranged to define pores having a pore size sufficiently small in the expanded state to promote further occlusion by a biological process on the expandable lattice.

28. The device of claim 27, wherein the expandable lattice defines a profile of the device intersecting the axis, the hub disposed on the axis at a distance of about 5 mm from the intersection of the profile and the axis.

29. The device of claim 27, wherein the expandable lattice defines a profile of the device intersecting the axis, the hub disposed on the axis at a distance of less than about 2 mm from the intersection of the profile and the axis.

30. The device of claim 27, wherein the device is free of any polymer component.

31. The device of claim 27, wherein the expandable lattice comprises a metallic outer lattice layer overlapping a metallic inner lattice layer.

32. The device of claim 31, wherein the device is free of any polymer component.

33. The device of claim 32, wherein the outer lattice layer is an occlusive lattice layer having wires with an average diameter less than 0.4 mm.

34. The device of claim 33, wherein the inner lattice layer is a structural lattice layer having wires with an average diameter between about 0.07 mm and about 0.20 mm.

35. The device of claim 34, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

36. A method of occluding a passage through cardiac or vascular tissue, the passage having opposing open ends, the method comprising:
expanding a first expandable occlusion member on one of the opposing open ends of the passage;
expanding a second expandable occlusion member on the other one of the opposing open ends of the passage; and
drawing the first and second occlusion members towards each other to occlude the passage.

37. The method of claim 36, further comprising:
expanding the first or second expandable occlusion members within the passage to occlude the passage.

38. A method of occluding a passage through tissue, the passage having at least one open end, the method comprising:
expanding a first expandable occlusion member to occlude the passage; and
expanding a second expandable occlusion member within the first expandable occlusion member.

39. The method of claim 38, further comprising:
expanding pores of the first or second expandable occlusion member to provide a pore size sufficiently small to promote further occlusion by a biological process.

40. The device of claim 3, wherein the inner lattice layer is a structural lattice layer having wires with an average diameter between about 0.07 mm and about 0.20 mm.

41. The device of claim 3, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

42. The device of claim 14, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

43. The device of claim 24, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

44. The device of claim 31, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

45. The device of claim 32, wherein the inner lattice layer is a structural lattice layer having wires with an average diameter between about 0.07 mm and about 0.20 mm.

46. The device of claim 32, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.

47. The device of claim 33, wherein the outer lattice layer is an occlusive lattice layer with wires having a first average diameter and the inner lattice layer is a structural lattice layer with wires having a second average diameter, a ratio of the second average diameter to the first average diameter being a range from 2:1 to 12:1.
